(19) United States
(12) Patent Application Publication
(10) Pub. No.: US 2012/0255558 A1
(43) Pub. Date: Oct. 11, 2012

(54) TISSUE GUARD FOR MEDICAL APPLICATIONS

(76) Inventor: Jose Luis Alvarez, El Paso, TX (US)

(21) Appl. No.: 13/079,855

(22) Filed: Apr. 5, 2011

(51) Int. Cl. A61B 19/00

(52) U.S. Cl. 128/846

(57) ABSTRACT

A tissue guard for medical applications. The tissue guard may include a tissue shielding portion having a central area and a peripheral area, and an attachment portion that is coupled to the shielding portion and adapted to couple to a medical tool. The attachment portion may include a first prong and a second prong, disposed such that the medical tool is received between the prongs.

### BACKGROUND

Medical operations are typically performed in areas of a patient's body where clearances are small and visibility is poor. Additionally, the tissues of the patient may get in the way of the operator's tools, further reducing the operator's visibility and increasing the risk of injury to the patient from the operation of the tool.

To alleviate these problems, the operator has to use a free hand to retract the intrusive tissues. In cases where this is not feasible, an assistant may use their hands or tools to retract the intrusive tissues as well. However, this requires substantial coordination between the operator and the assistant, and inhibits the assistant from carrying out other activities necessary for the operation.

### SUMMARY

According to at least one exemplary embodiment, a tissue guard for medical applications is provided. The tissue guard may include a tissue shielding portion having a central area and a peripheral area, and an attachment portion that is coupled to the shielding portion and adapted to couple to a medical tool. The attachment portion may include a first prong and a second prong, disposed such that the medical tool is received between the prongs.

In other exemplary embodiments, the attachment portion may be removably coupled to the shielding portion. Furthermore, the distance between the prongs may be varied so as to accept medical tools of varying sizes. The shielding portion may also have various configurations or shapes and may include a cushion or an absorbent covering.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

FIGS. 1a-1f show a first exemplary embodiment of a tissue guard.

FIGS. 2a-2b show a second exemplary embodiment of a tissue guard.

FIGS. 3a-3b show a third exemplary embodiment of a tissue guard.

FIGS. 4a-4b show an exemplary embodiment of a tissue guard in use.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiment are not necessarily to be construed as preferred or advantageous over other embodiments. Moreover, the terms “embodiments of the invention”, “embodiments” or “invention” do not require that all embodiments of the invention include the discussed feature, advantage or mode of operation.

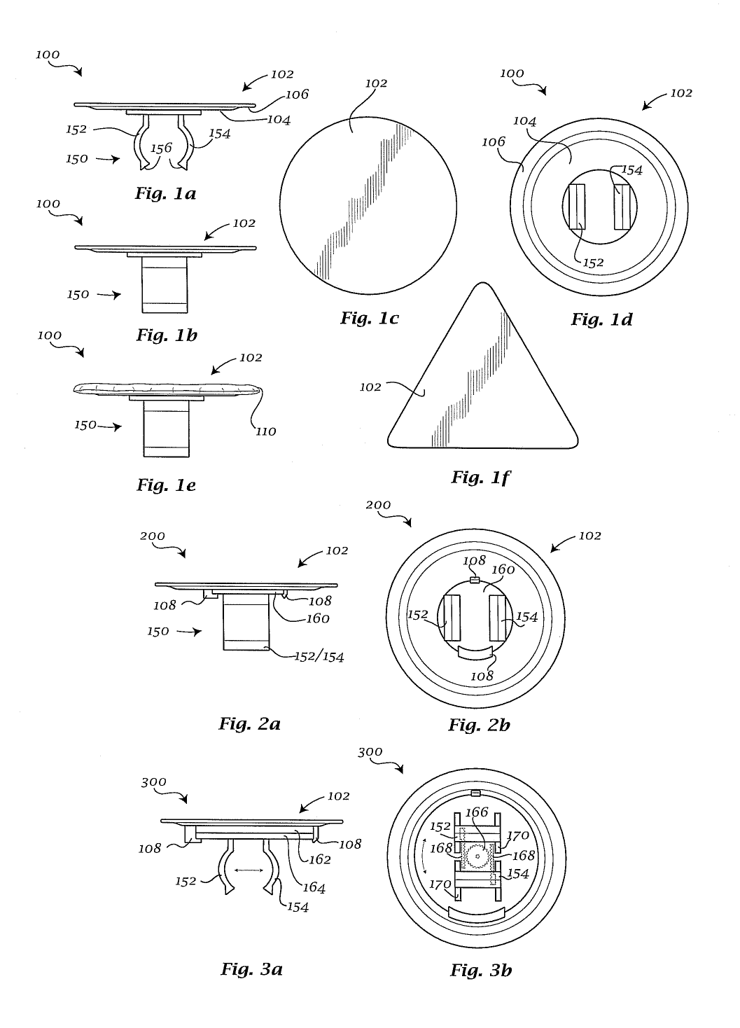

According to at least one exemplary embodiment, and generally referring to FIGS. 1a-1f, a tissue guard 100 is disclosed. The tissue guard can include a shielding portion 102 and an attachment portion 150 coupled to the shielding portion. Tissue guard 100 may be attached to a medical tool or device, for example a dental power tool, by fitting attachment portion 150 onto the body of the medical tool. The medical tool may then be inserted into a particular location or cavity, for example a patient's mouth. While the operator of the medical tool is performing the necessary procedures in the particular location, tissue guard 100 can facilitate maintaining the patient's tissues at a distance from the medical tool. This can allow the operator to have easier control of the medical tool and can further provide the operator with a clear view of the area being operated upon. The use of tissue guard 100 can further allow the operator to perform the necessary procedures without having an assistant retracting the patient's tissues away from the dental tool.

Shielding portion 102 may have a substantially circular or oval shape. Alternatively, shielding portion 102 may have a substantially triangular shape, a substantially rectangular shape, an irregular shape, or a shape having a contour that is adapted to fit in a particular location or cavity, for example, in a portion of a patient's mouth. Shielding portion 102 may be formed from any desired material, for example a rigid material, a flexible or semi-rigid material, a resiliently deformable material, or any other material known in the art that enables tissue guard 100 to function as described herein. Shielding portion 102 may further include a central area 104 and a peripheral area 106. Peripheral area 106 may have a thickness that is smaller than the thickness of central area 104. The smaller thickness of peripheral area 106 can facilitate the resilient deformation of peripheral area 106 when shielding portion 102 is exerting a force on a patient's tissue. This can serve to decrease the patient's discomfort by providing an amount of “give” to the shielding portion, thereby providing a softer edge to shielding portion 102 and reducing pressure on the patient's tissues at the edges of shielding portion 102.

Attachment portion 150 may be adapted to couple to a portion of a medical tool, for example the shaft of the medical tool. To that end, attachment portion 150 may include a coupling structure that allows for easy attachment and removal of tissue guard 100. In one embodiment, the coupling structure may include a first prong 152 and a second prong 154. Prongs 152, 154 may each have an arcuate profile and may together define a cylindrical passage therebetween wherein a shaft of a medical tool may be received. The midpoint of the distance between prongs 152, 154 may be disposed over the center of tissue guard 100. Alternatively, prongs 152, 154 may have any desired shape that allows tissue guard 100 to function as described herein. Prongs 152, 154 may further have distal ends 156 that may be shaped so as to allow tissue guard 100 to be easily snapped onto the shaft of the medical device. To detach tissue guard 100 from the medical device, the prongs may be slightly pulled apart by the user so as to allow the shaft of the medical device to be removed from therebetween. Alternatively, tissue guard 100 may be attached and detached from the medical device by placing attachment portion 150 at an end of a shaft of the medical device and sliding tissue guard 100 longitudinally along the shaft until tissue guard 100 is located in the desired position.

Shielding portion 102 may further be provided with a covering 110, as shown in FIG. 1e. Covering 110 may be disposed over shielding portion 102 and may include various materials. For example, covering 110 may include a padding material to reduce the patient's discomfort by providing a cushion between shielding portion 102 and the patient's tissue. Covering 110 may also include an absorbent material, for example gauze or the like, which can facilitate soaking up liquids in the particular location where the necessary operations are being performed. Covering 110 may be removable and disposable, and may be provided in sterile packaging.

In another exemplary embodiment of tissue guard 200, and as shown in FIGS. 2a-2b, attachment portion 150 may be detachable from shielding portion 102. This can allow the user to utilize a plurality of shielding portions, for example shielding portions having diverse shapes, such that the shape of the shielding portion is most appropriate for the particular procedure that is being undertaken. Furthermore, low-cost and disposable shielding portions 102 may be provided. Such low-cost and disposable shielding portions may further be provided in sterile packaging.

To facilitate easily attaching and detaching attachment portion 150 from shielding portion 102, attachment portion 150 may further include a base 160 to which prongs 152, 154 may be coupled. Base 150 may have any desired shape or configuration, and may have a diameter less than that of central area 104 of shielding portion 102. To further facilitate coupling of attachment portion 150 to shielding portion 102, shielding portion 102 may be provided with tabs 108 which are adapted to hold attachment portion 150 in place. Alternatively, various coupling arrangements may be utilized, for example hook-and-loop fasteners, snaps, or any other coupling arrangement that enables tissue guard 100 to function as described herein.

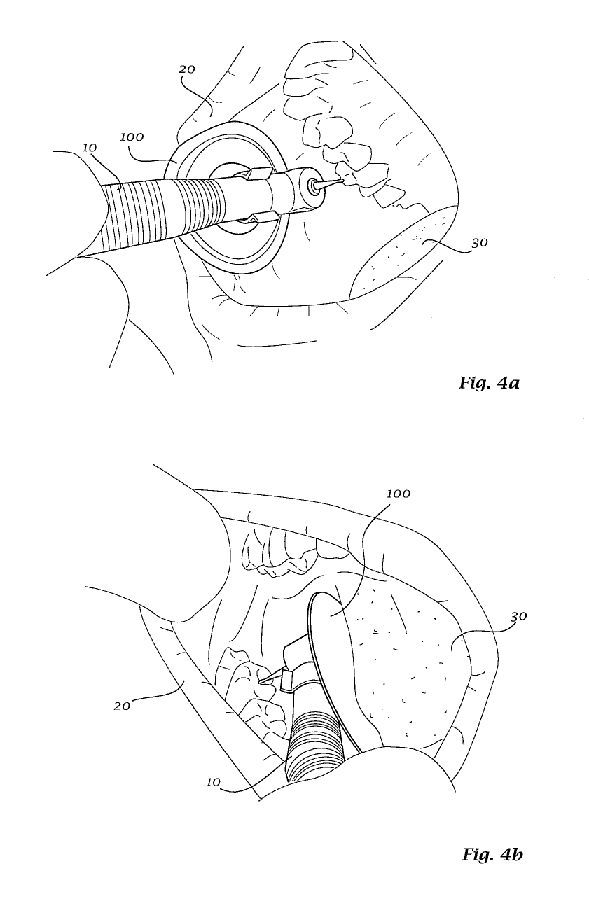

In another exemplary embodiment of tissue guard 300, and as shown in FIGS. 3a-3b, the distance between prongs 152, 154 may be variable and user-adjustable. For example, base 160 may include a first portion 162 rotatably coupled to a second portion 164. Disposed within base portion 160 may be a rack and pinion mechanism having a central gear 166 and a pair of pinions 168, each of which can be coupled one of prongs 152, 154. Slits 170 may be provided in second portion 164 such that prongs 152, 154 may translate along the length of slits 170. Gear 166 may be fixedly coupled to first portion 162 of base 160. Thus, when second portion 164 is rotated with respect to first portion 162, the distance between prongs 152, 154 may easily be varied, and the midpoint of the distance between the prongs may be maintained over the center of tissue guard 100. Alternatively, any other known arrangement for varying the distance between prongs 152, 154 may be provided.

FIGS. 4a-4b show an embodiment of tissue guard 100 in operation and coupled to a dental tool 10. As shown in FIG. 4a, using tissue guard 100 in conjunction with dental tool 10 can allow the user to operate on the teeth from the outside of the jaw without using a second hand, an assistant, or a separate tool to retract the lip 20 of the patient. The user is also provided with an increased field of vision into the operating area. Furthermore, as shown it FIG. 4b, using tissue guard 100 in conjunction with dental tool 10 can allow the user to operate on the teeth from the inside of the jaw without using a second hand, and assistant, or a separate tool to retract the tongue 30 of the patient. It should further be appreciated that the uses of tissue guard 100 are not limited to the scenarios described herein and depicted in the figures; rather the uses of tissue guard 100 may include any situation where additional clearance is needed between a medical tool and a patient's tissue, or any similar or analogous situation that is known in the art.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A guard for a medical tool, comprising:
a shielding portion comprising a central area and a peripheral area; and
an attachment portion coupled to the shielding portion and adapted to couple to a medical tool.

2. The guard of claim 1, wherein:
the attachment portion further comprises a first prong and a second prong; and
wherein the medical tool is received between the first prong and the second prong.

3. The guard of claim 2, wherein:
the first prong has an arcuate profile; and
the second prong has an arcuate profile.

4. The guard of claim 1, wherein the attachment portion is removably coupled to the shielding portion.

5. The guard of claim 2, wherein the distance between the first prong and the second prong is adjustable.

6. The guard of claim 1, wherein the peripheral area of the shielding portion is resiliently deformable.

7. The guard of claim 4, wherein the shielding portion is disposable.

8. The guard of claim 1, further comprising a covering disposed over the shielding portion.

9. The guard of claim 8, wherein the covering is a cushion.

10. The guard of claim 8, wherein the covering is absorbent.

11. The guard of claim 1, wherein the shielding portion is substantially circular.

12. The guard of claim 1, wherein the shielding portion is substantially triangular.
